(19) United States
(12) Patent Application Publication
(10) Pub. No.: US 2012/0071955 A1
(43) Pub. Date: Mar. 22, 2012

(54) THERMAL PACK HOLDING DEVICE AND METHOD FOR RELIEF OF BREAST PAIN OR DISCOMFORT

(76) Inventor: PAULA B. YOCKEL, Jones, OK (US)

(21) Appl. No.: 13/077,000

(22) Filed: Mar. 31, 2011

Related U.S. Application Data
(60) Provisional application No. 61/383,417, filed on Sep. 16, 2010.

(51) Int. Cl. A61F 7/00

(52) U.S. Cl. 607/114

(57) ABSTRACT

A thermal pack holding device and method of using same. The thermal pack holding device holds two or more thermal packs (cold packs or hot packs) for providing comfort and pain relief to the breasts of the user. The thermal pack holding device is constructed and used in such a manner as to minimize breast compression thereby preventing cessation of lactation and plugged milk ducts resulting from compression of the breasts.

### CROSS-REFERENCE TO RELATED APPLICATIONS

The present application claims the benefit under 35 U.S.C. 119(e) of U.S. Provisional Application Ser. No. 61/383,417, filed Sep. 16, 2010, the entirety of which is hereby expressly incorporated herein by reference.

### BACKGROUND

Breast discomfort or pain is a common outcome of post partum lactation. During pregnancy, the mother's breasts undergo changes including vein expansion and glandular (alveoli) development. Late in pregnancy, and after birth, the mother's breasts produce colostrum, a yellow fluid which provides concentrated nutrition which is high in proteins (antibodies) which provide passive immunity for protecting the newborn from disease, as well as many components and factors which aid in the development of the baby's innate immune system and the gut. Colostrum is considerably lower in fats and sugars than normal milk thus delivers its nutrients in a low-volume form. However, within a few days after birth, breast production switches from colostrum to mature milk. Amid fat cells and glandular tissue within the breasts are milk ducts, an intricate network of channels that grow in number and size during pregnancy and whose purpose is to deliver milk to the newborn baby. As milk production and thus volume increases rapidly, the capacity of the breast to store the milk in the alveolar glands, where the milk is produced, may be exceeded. The accumulation of milk in the breast, if not alleviated by nursing and/or pumping, can result in breast engorgement, which may in turn lead to breast pain and even damage to the alveoli and nipple inversion thereby causing even more pain, stagnation of milk within the breast, and cessation of milk production. Unrelieved severe engorgement can even lead to plugged milk ducts and infection of breast tissue (mastitis). Plugged milk ducts are a condition that occurs when the milk ducts do not properly drain and become inflamed. Pressure then builds up behind the plugged ducts and the surrounding tissue becomes inflamed. Breast engorgement and plugged milk ducts typically result in significant discomfort and even pain, for a matter of days to weeks. Symptoms of breast engorgement may include a gradually rising body temperature, pain and tenderness in one or both breasts, edematous and flushed nipples, and even nipple retraction, loss of appetite, fatigue, weakness, and chills. Symptoms of plugged milk ducts may include sore and tender lumps in the breast. It is known and accepted that compression or constriction to the breast while lactating may induce cessation of lactation as well as the creation of plugged milk ducts which can result in mastitis.

As is evident, effective means for the alleviation of pain and discomfort due to breast engorgement and/or plugged milk ducts is highly desired by women who suffer from these conditions. Various pain medications such as ibuprofen provide some relief, however, results are often delayed, and there are limits to the quantities and frequency of use of such medications. It is known that ice packs or cold packs can provide a degree of immediate relief and there have been a number of devices put forth before the present invention which are explicitly designed for, or could be used for supporting ice packs or cold packs (or even heat packs) near or on the breasts. Such devices, as well as other devices which relate to thermal treatment of various body parts, or which relate to other means of support or treatment of the breasts are described below.

U.S. Pat. No. 4,527,566 discloses a body wrap, intended to snugly apply heat therapy to a body portion, which requires greater length (top to bottom) than width (side to side) such as a leg or arm. This design and method is deficient for applying cold and heat therapy in a non-compression manner to the female human breast(s). The flat shape and dimensional ratio of the thermal area, as well as the coverage area are all incompatible with the engorged female breast anatomy. Additionally, the intended compression in this design is not conducive to continued lactation for a breastfeeding mother and may lead to plugged milk ducts.

U.S. Pat. No. 4,676,247 discloses a multi-pocket thermal wrap for a joint of the human body, wherein the pockets are sized, dimensioned, placed within the device, and intended for coverage area of human joints, not human breasts. Additionally, the apparatus imposes compression on the affected area due to its means of affixing to the affected area. The pockets intended for containing the thermal therapy are inadequate in size, dimension, placement within the device and intended coverage area for applying thermal therapy in a “surrounding and conforming manner” to engorged breasts. The compression caused by the device's means of attachment would be uncomfortable and counterproductive in an application used for engorged breasts and may result in plugged milk ducts. This device has material and binding hardware extending outward from the thermal holding pockets towards each end of the device which create bulk that would be uncomfortable and cumbersome if this device was attempted to be used as thermal therapy on engorged breasts.

U.S. Pat. No. 4,960,112 discloses a breast binder having shoulder and back straps for wrapping about the breasts for causing compression of the breasts with the express purpose of causing suppression of lactation of the breasts. The device does not include any kind of thermal packs.

U.S. Pat. No. 5,050,595 discloses a device which is limited to applying heat therapy to breasts and is not for application of cold therapy to breasts. The design of the device replicates a traditional brassiere which has back and shoulder straps and which is very size specific, therefore does not accommodate an array of breast sizes. This device requires activation of a power supply to heat the thermal unit, which limits the application of the device and complicates the apparatus. A new mother that is breastfeeding is most likely to wear a nursing bra and does not want the complexity of having to change into an alternate bra to apply thermal therapy for relief of breast engorgement. Similarly, it would not be feasible, nor advisable, to attempt to wear both a nursing bra and thermal therapy bra at the same time. This device is form-fitting and compression-causing which is not desired in a mother who is suffering from engorgement pain and wishes to continue breastfeeding. The compression which results from this design is not conducive to continued lactation for a breastfeeding mother and may lead to plugged milk ducts.

U.S. Pat. No. 5,060,648 discloses a breast binding device which is intended to apply iced compression to the breasts of a mother who no longer intends to breastfeed. The device has a back panel which is tightened about the back of the user. The compression of this device is counterproductive to mothers who wish to apply cold or heat therapy to for relief of breast engorgement pain but who do not wish to cause the lactation process to be reduced or stopped altogether. The compression resulting from this device may lead to plugged milk ducts which are an undesired condition for the lactating breast.

U.S. Pat. No. 5,427,563 discloses a breast wrap which is intended to apply iced compression to the breasts for purposes of stopping lactation in a woman after child birth. The compression caused by this device is counterproductive for mothers who wish to apply cold or heat therapy to for relief of breast engorgement pain, but who wish to continue to be able to breastfeed. As is well known in the art, compression of lactating breasts leads to cessation of lactation and may also lead to plugged milk ducts. If one chose to use this device in a less compressing manner by not adjoining the hook and loop fasteners, the fabric of the device intended to surround the body would create unnecessary and uncomfortable bulk and potential discomfort from the attachment means.

U.S. Pat. No. 5,507,794 teaches a therapeutic support garment which is intended to encircle the torso of the wearer and employ an attaching means to hold thin thermal gel packs onto one or two breasts which could result in compression of the breasts as the thermal therapy is applied. The device is secured about the torso by attaching means. The resulting compression is counterproductive for nursing mothers who desire to continue breastfeeding as compression to the breasts is one means of reducing and/or eliminating breast milk production as noted elsewhere herein and compression to the breasts may lead to plugged milk ducts. Additionally, the apparatus, while claiming to accommodate a wide range of upper torso sizes is actually limited in its range, extending only to the size accommodated by the limited length of the support member to which the breast pouch(es) are attached. The breast pouches of the apparatus intended for holding thermal packs are designed to hold thin gel packs in order to achieve a maximum coverage of the breast area of 6″×6″. This limitation restricts the wearer's choice of thermal pack devices. The breast pouches are affixed to the “positioning member” of the apparatus which is a fibrous hook material that could potentially be scratchy against the wearer's body.

U.S. Pat. No. 5,716,388 discloses a flexible pouch device which is limited to applying heat therapy to a single area of the body (e.g., a knee), not two areas simultaneously (i.e., not both breasts). Additionally, the single strap attachment mechanism applies pressure in the vertical center of the thermal pack which then comprises the part of the body on which it is applied. This would not enable a conforming fit around the curvature of a breast. Additionally, compression created by the attachment mechanism is counterproductive to the treatment of breast engorgement for mothers desiring to breastfeed and the compression may lead to plugged milk ducts. This device is intended only for heat therapy.

U.S. Pat. No. 5,839,942 discloses a Post-Partum Breast Engorgement Bra which requires the user to wear it as a brassiere in order to benefit from the thermal therapy. The bra has back and shoulder panels or straps designed to hold the bra tightly against the user's body. A new mother that is breastfeeding is most likely to wear a nursing bra and does not want the complexity of having to change into an alternate bra to apply thermal therapy for relief of breast engorgement. Similarly, it would not feasible, nor advisable to attempt to wear both a nursing bra and thermal therapy bra at the same time. Further, compression caused by the straps of the bra is counterproductive to continued lactation and may lead to plugged milk ducts. The design of the device replicates a traditional brassiere which is very size specific, therefore does not accommodate an array of breast sizes.

U.S. Pat. No. 6,394,879 is a brassiere which is intended to provide postpartum breast relief to mothers that do not breastfeed. The device has back and shoulder panels and is constrictive and compression-causing by design while also applies thermal therapy to a limited area of each breast. It is counterproductive for a woman to wear such a restrictive compressive bra if she wants to continue breastfeeding as well as avoid plugged milk ducts resulting from the constrictive bra. Further, she is most likely to wear a nursing bra, and would not want the complexity of having to change into an alternate bra to apply thermal therapy for relief of breast engorgement. Similarly, it would not feasible, nor advisable to attempt to wear both a nursing bra and thermal therapy bra at the same time.

U.S. Pat. No. 6,464,717 discloses a bra with Hot/Cold Inserts. Again, this device has shoulder and back straps which cause compression. The deficiency of this device is that it requires wearing it as a brassiere to in order to benefit from the thermal therapy. A new mother that is breastfeeding is most likely to wear a nursing bra and does not want the complexity of having to change into an alternate bra to apply thermal therapy for relief of breast engorgement. Similarly, it would not be feasible, nor advisable, to attempt to wear both a nursing bra and thermal therapy bra at the same time. This device is form-fitting and compression-causing which is not desired in a mother who is suffering from engorgement pain but wishes to continue breastfeeding. Additionally, plugged milk ducts may result from the constrictive nature of the device. This device requires specific fitting due to its brassiere-type method and one size does not accommodate a broad range of body sizes, girth measurements, nor breast sizes.

U.S. Pat. No. 6,936,018 discloses a therapeutic wrapping device for wrapping a body part such as an arm, elbow, knee, or shoulder. This device applies heat therapy and compression to one area of the body (e.g., a thigh), not two (e.g., two breasts). The compression created by the attachment mechanism is not desired in the treatment of breast engorgement for mothers desiring to breastfeed and the compression may result in plugged milk ducts. It is unclear how the device could surround the torso and two engorged breasts without causing compression leading to the cessation of lactation and/or plugged milk ducts.

U.S. Pat. No. 7,275,977 is a bra with back and shoulder panels and is intended to apply hot or cold therapy to breasts or ribs with significant compression for relief of post surgery pain. It is not intended to provide relief from breast engorgement pain. The base structure of this device is a sports bra which by design is intended to be tight and restrictive. A sports bra is applied by the wearer putting her head and arms through tight elastic openings in the garment, then stretching the garment over the breasts and onto the rib cage into its resting position where it sits securely and tightly in place. This is not a feasible or desired activity for a new mother suffering from the pain of lactation-related breast engorgement or breastfeeding tenderness because the tight fit and related compression of the device will impair or eliminate lactation in a breastfeeding mother and may result in plugged milk ducts. The device, being a brassiere-type method, requires size fitting and does not cover a large range of body sizes, body girth measurements, nor breast sizes.

U.S. Pat. No. 7,309,275 discloses a therapeutic brassiere incorporating side and back panels and thermal gel packs. The user is required to wear it as a brassiere in order to benefit from the thermal therapy. A new mother that is breastfeeding is most likely to wear a nursing bra and does not want the complexity of having to change into an alternate bra to apply thermal therapy for relief of breast engorgement. Similarly, it would not feasible, nor advisable to attempt to wear both a nursing bra and thermal therapy bra at the same time. Further, this device is form-fitting and causes breast compression which is not desired in a mother who is suffering from engorgement pain, but wishes to continue breastfeeding. As noted elsewhere, compression of the breast may also lead to plugged milk ducts. This device requires specific fitting due to its brassiere-type method and one size does not accommodate a broad range of body sizes, girth measurements, nor breast sizes.

Clearly, there is a need for a comfortable device for providing thermal relief to engorged breasts, and which does not cause compression which would lead to the premature stoppage of lactation and/or plugged milk ducts in a breastfeeding mother. It is to this need that the present invention is directed.

### BRIEF DESCRIPTION OF THE DRAWINGS

FIG. 1 is a perspective view of a thermal pack holding device of the present invention.

FIG. 2 is a perspective view of the fabric components used in one embodiment to construct the thermal pack holding device of FIG. 1.

FIG. 3 is a top plan view of the thermal pack holding device of the present invention.

FIG. 4 is a longitudinal cross-sectional view of the thermal pack holding device of FIG. 1 wherein a pair of thermal packs are disposed within a pair of pockets of the holding device.

FIG. 5 is a perspective view of a user having the thermal pack holding device positioned upon the breasts for alleviation of breast discomfort or pain in accordance with the present invention.

FIG. 6 is a perspective view of an alternate embodiment of the present invention wherein the thermal pack holding device has fastening means for closing the openings of the pockets after thermal packs have been disposed therein.

FIG. 7 is a cross-section view of an embodiment of the thermal pack holding device of the present invention comprising flaps as a component of the fastening means for closing the pockets.

FIG. 8 is a top plan view of an embodiment of the thermal pack holding device of the invention wherein the fastening means for closing the pockets are positioned on opposite ends of the device.

FIG. 9 is a top plan view of an embodiment of the thermal pack holding device of the invention wherein the fastening means for closing the pockets are both positioned along a longitudinal edge of the device, such as the upper edge.

FIG. 10 is a top plan view of an embodiment of the thermal pack holding device of the present invention wherein the shape of the device is rounded on the corners and wherein a medial (central) position of the device is narrowed in relation to the width of the pockets.

FIG. 11 is a cross-sectional view of an alternate embodiment of the invention wherein the thermal pack holding device has a pair of concavities under the pockets such that the shape of the device is adapted to conform to the shape of the breasts.

FIG. 12 is a top plan view of an embodiment of the thermal pack holding device of the present invention wherein the opening into each pocket is along or near a medial portion of each pocket.

FIG. 13 is a cross-sectional view of the thermal pack holding device of FIG. 12.

### DETAILED DESCRIPTION OF THE INVENTION

The invention described herein is a thermal pack holding device (commercially referred to as COMFORT POCKETS™), and a method of using the same. The device is preferably fabricated at least partially of a non-elastic cotton flannel fabric and is intended for use by a woman to aid in the relief of breast discomfort or pain associated with, for example, post-partum breast engorgement and/or breastfeeding and/or plugged milk ducts. The thermal pack holding device may be used or medically prescribed for other therapeutic purposes associated with breast surgeries, breast maladies, hormonal conditions and other conditions which result in breast discomfort or pain. In use, the device holds a pair of thermal packs thereby forming a thermal pack system which is placed onto the breasts preferably when the user is in a fully- or semi-reclined position as described in further detail below. While the thermal pack holding device of the invention is constructed in a preferred embodiment to hold two thermal packs, the device optionally may be constructed to hold a plurality of thermal packs, including but not limited to 3, 4, 5, 6, 7 8 or 9 pockets each for holding a thermal pack. The thermal pack holding devices of the invention are preferably made of soft, natural, 100% cotton flannel fabric and generally have a rectangular (or other) shape, as described below in more detail. In a preferred embodiment the thermal pack holding device of the present invention completely covers the breasts of the user.

Compressing or constricting the breasts in lactating women is well known to be a factor in causing cessation of lactation, plugged milk ducts and/or mastitis, therefore the device of the present invention is intended to cause no sustained compression to the breasts when applied thereto or disposed thereon except to the extent compression is applied by the weight of the device and thermal packs loaded therein and therefore, in preferred embodiments, the device is absent restraining means such as straps, belts, webbing or other restraining devices which are intended to cause, effect, or enhance breast compression when the device is applied to the body of the user, or which are intended to secure the device to the user's body when the user is in an upright orientation, for example, in the configuration of a brassiere.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples, which include preferred embodiments, will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

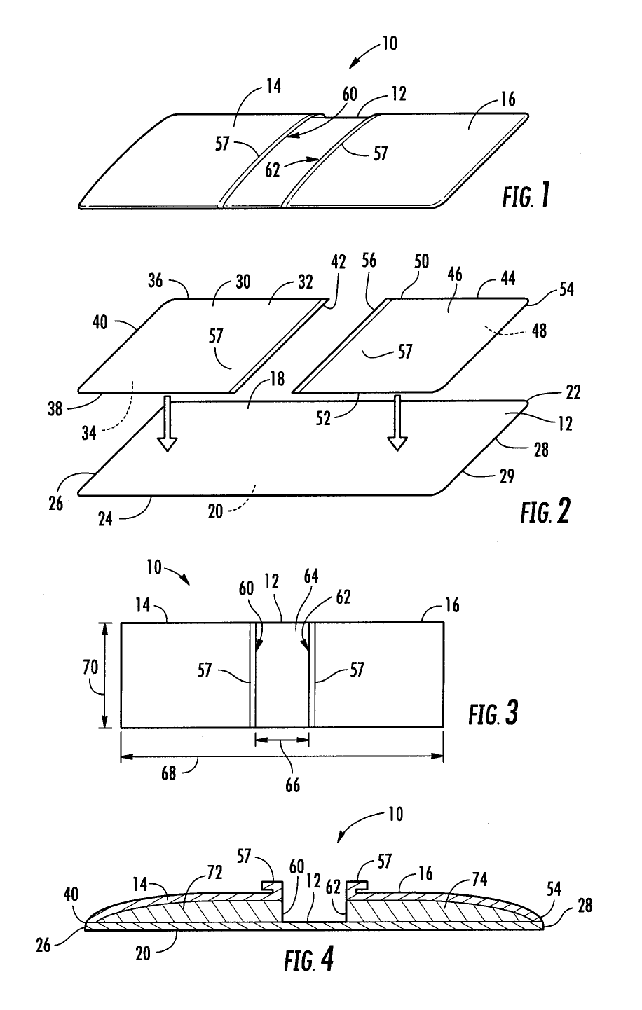

Turning now to the drawings, shown in FIGS. 1 to 5, and referred to therein by the general reference numeral 10, is a thermal pack holding device. Thermal pack holding device 10 comprises a base panel 12, a first pocket 14, and a second pocket 16. The base panel 12 has, as shown in FIG. 2 an upper surface 18, a lower surface 20, an upper edge 22, a lower edge 24, a first side edge 26 and a second side edge 28. Together the upper edge 22, lower edge 24, first side edge 26, and second side edge 28 comprise an outer perimeter 29. Preferably the base panel 12 is constructed of flannel (cotton) fabric but may be constructed of any soft natural or synthetic fabric which functions in accordance with the present invention. For example, the base panel 12 may be constructed of one or more fabrics, such as but not limited to, linen, rayon, silk, nylon, in addition to, or in substitution of, cotton. The base panel 12 may be constructed of a blend of such materials. The key requirement of the material used to fabricate the base panel 12 is that it be soft and flexible, comfortable, non-irritating, and non-abrasive when resting on the breasts of the user. The thermal pack holding device 10, due to the flexibility of the material used in its construction, is able to substantially conform to the shape of the user's breasts. In the embodiment of FIGS. 1-5, the first pocket 14 is constructed of a piece of fabric which is the same as or different from the material used to construct the base panel 12. The first pocket 14 is constructed from a first pocket panel 30 which has an upper surface 32, a lower surface 34, an upper edge 36, a lower edge 38, an outer side edge 40, and an inner side edge 42. The second pocket 16 is constructed of a piece of fabric which is (as for first pocket 14) the same as or different from the material used to construct the base panel 12. The second pocket 16 is constructed from a second pocket panel 44 which has an upper surface 46, a lower surface 48, an upper edge 50, a lower edge 52, an outer side edge 54, and an inner side edge 56. In a preferred version of the invention, each inner side edge 42 and 56 has a hem 57 which preferably is a rolled hem. The hem 57 is optional. The thermal pack holding device 10 is then constructed by attaching the first pocket panel 30 and the second pocket panel 44 to the base panel 12 (as shown in FIGS. 1 and 2). The first pocket panel 30 is preferably attached to the base panel by stitching, such as serger stitching, such that the outer side edge 40 is positioned near the first side edge 26, the upper edge 36 is positioned near the upper edge 22, and the lower edge 38 is positioned near the lower edge 24. The inner side edge 42 is left unstitched to the base panel 12 forming a first pocket opening 60 (see FIG. 1). Similarly, the second pocket panel 44 is attached to the base panel 12, preferably by stitching, such that the outer edge 54 is positioned near the second side edge 28, the upper edge 50 is positioned near the upper edge 22, and the lower edge 52 is positioned near the lower edge 24. The inner side edge 56 is left unstitched to the base panel 12 forming a second pocket opening 62 (FIG. 1). In another method of construction, the device 10 may be formed from a single piece of material wherein the first pocket panel 30 and second pocket panel 44 are integral portions of the base panel 12 and extend from the ends of the base panel 12 and are simply folded over the base panel 12 and are attached on two of the three sides thereof leaving an opening for each of the two pockets formed from the first and second pocket panels 30 and 44, respectively.

As shown in FIG. 3, there is a gap 64 between first pocket opening 60 (and inner side edge 42) and second pocket opening 62 (and inner side edge 56). The gap 64 however is optional and the inner side edge 42 of the first pocket 14 may be substantially adjacent to the inner side edge 56 of the second pocket 16. The gap 64, when present, has a width 66 which is generally in a range of about 1 inch to about 5 inches, with width 66 preferably about 2.5 to 4.0 inches, and most preferably about 3 to 3.5 inches. The thermal pack holding device 10 in a preferred embodiment, as shown in FIGS. 1-5, has a rectangular shape having a length 68 and a width 70. Preferably the length 68 is at least 2 times and up to about 4 times greater than the width 70 (2:1 to 4:1 ratio), and more preferably 2 times to 3.5 times greater (2:1 to 3.5:1 ratio), and most preferably 2 to 3 times greater (2:1 to 3:1 ratio), that is, at least 2.0, to 2.1, to 2.2, to 2.3, to 2.4, to 2.5, to 2.6, to 2.7, to 2.8, to 2.9, to 3.0 times greater. A length 68 to width 70 ratio of 2.6 to 2.8 is preferred, and a ratio of about 2.7 most preferred.

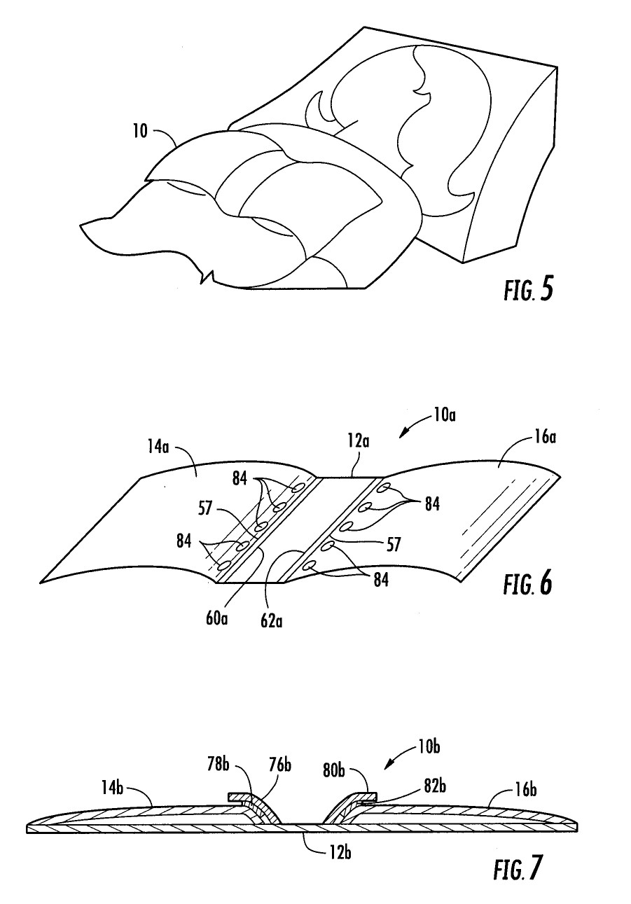

Shown in FIG. 4 is a thermal pack system comprising the thermal pack holding device 10 of FIG. 1 which has a first thermal pack 72 disposed within first pocket 14 and a second thermal pack 74 disposed within second pocket 16. The first thermal pack 72 and second thermal pack 74 are loaded within the pockets 14 and 16, respectively, simply by lifting a portion of the openings 60 and 62 of the pockets 14 and 16, respectively, and inserting each first and second thermal pack 72 and 74 through the corresponding opening. The user, in a preferred embodiment, then reclines in a prostrate or partially inclined position and rests the loaded thermal pack holding device 10 on her breasts in a position such that the loaded thermal pack holding device 10 (i.e., the thermal pack system) will not slide readily from her chest, for example as shown in FIG. 5.

While the first and second pocket panels 30 and 44 are preferably permanently secured to the base panel 12 by stitching, alternatively they may be removably secured thereto, for example by snaps, buttons, zippers, ties, hook and loop fasteners (e.g., VELCRO®), pins, or other fastening or closure means known in the art for removably securing items together.

Shown in FIG. 6 is an alternate embodiment of the thermal pack holding device of the present invention which is designated by the general reference numeral 10a and which has fastening means 84 along only the margins of first and second pocket openings 60a and 62a for enabling closure of pockets 14a and 16a, respectively after thermal packs (e.g., first and second thermal packs 72 and 74) have been disposed therein. The fastening means 84 may be, as noted above, zippers, buttons, ties, snaps, hook and loop fasteners, or any other appropriate fastening device or attaching device known in the art.

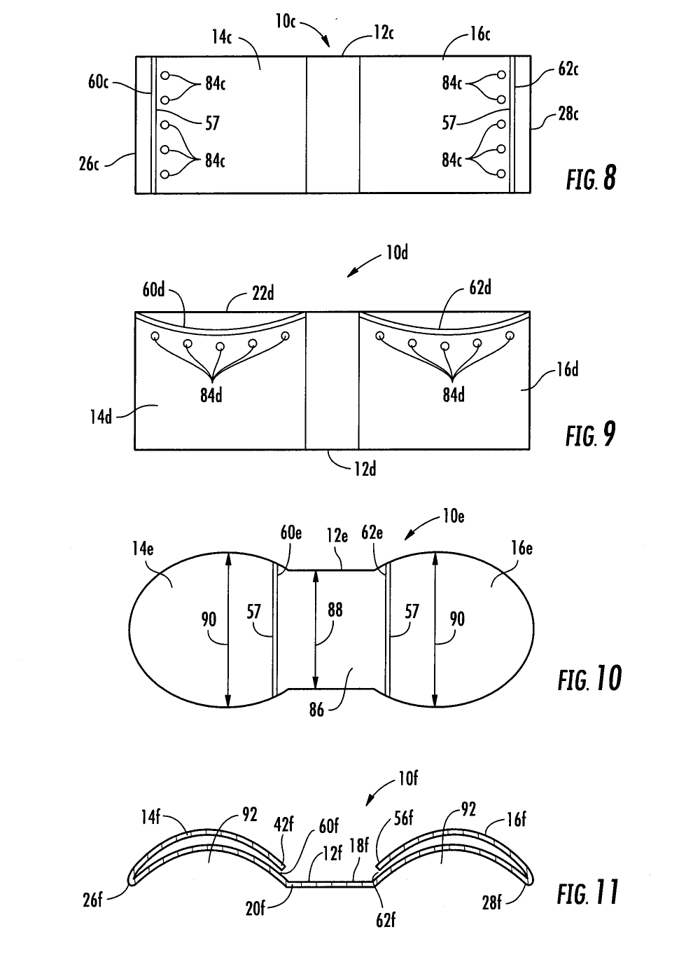

Shown in FIG. 7 is an alternate embodiment of the present invention referred to therein by the general reference numeral 10b. Thermal pack holding device 10b comprises a first pocket 14b and a second pocket 16b. The first pocket 14b is closeable via a fastening means comprising a flap 76b having a hook and loop closure device 78b (or any other fastening means described herein), and the second pocket 16b is closeable via a fastening means comprising a flap 80b having a hook and loop closure device 82b (or any other fastening means described herein).

While it is preferred that the first pocket openings 60 and 60a of the first pockets 14 and 14a, respectively, and second pocket openings 62 and 62a of the second pockets 16 and 16b, respectively, face each other as shown in FIGS. 1-6, the opening of the pockets may be located on opposite ends, or along upper or lower edges of the base panels 12 and 12a, respectively. For example, FIG. 8 shows a thermal pack holding device 10c which has a first pocket 14c with an opening 60c with fastening means 84c which are adjacent a first side edge 26c, and a second pocket 16c with a second pocket opening 62c with fastening means 84c which are adjacent a second side edge 28c, wherein the openings 60c and 62c are on opposite ends of the thermal pack holding device 10c.

FIG. 9 shows a thermal pack holding device 10d which has a first pocket 14d with a first pocket opening 60d with fastening means 84d which are adjacent an upper edge 22d of a panel 12d and a second pocket 16d with a second pocket opening 62d with fastening means 84d which are adjacent the upper edge 22d of panel 12d. Fastening means 84d of first and second pocket openings 60d and 62d are thus substantially parallel to the same upper edge 22d of panel 12d. Similarly, in yet another embodiment of the invention, the openings and fasteners of the pockets may be along a lower edge of the base panel of the thermal pack holding device. Where used herein, the term “fastening means” is intended to refer to any fastening or closure means described or discussed herein, including snaps, buttons, ties, zippers, hook and loop fasteners, flaps (as shown in FIG. 7), or other fastening means known to persons of ordinary skill in the art. For example, even though a plurality of fastening means are referred to by the reference numeral 84c in FIG. 8, each first and second pocket opening 60c and 62c therein may be closed by a single fastener such as a snap, button, tie, zipper, flap, hook and loop device, or other fastener.

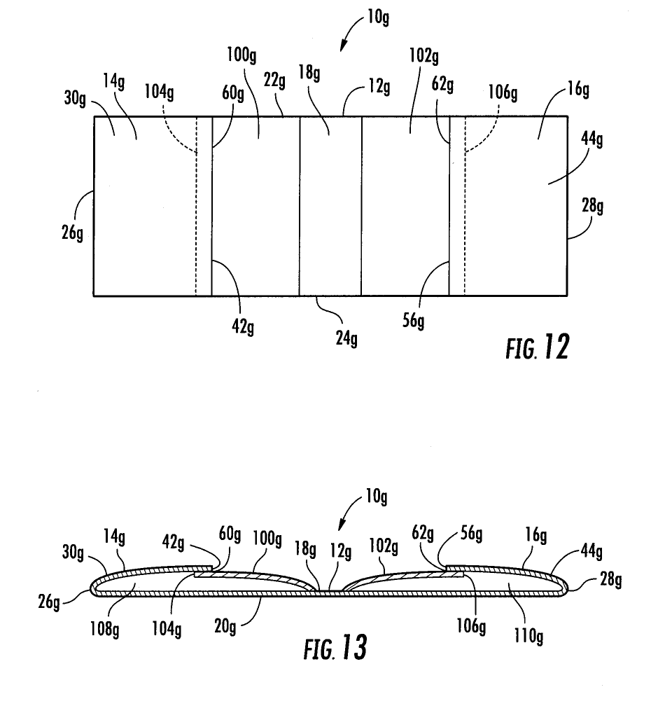

The thermal pack holding devices 10-10d are shown as having substantially rectangular shapes. However any of the thermal holding devices described herein may have rounded corners, for example as shown in an embodiment of the invention represented in FIG. 10, which shows a thermal pack holding device 10e. The thermal pack holding device 10e is the same as any of the other thermal pack holding devices shown herein except the thermal pack holding device 10e has a base panel 12e which has a binary “oval” or “rounded” shape with curved outer edges, and has first and second pockets 14e and 16e, respectively, which conform to the oval or rounded portions of the base panel 12e. In this embodiment, the base panel 12e has a central portion 86 which has a minimum width 88 which is less than a maximum width 90 of the oval or rounded first pocket 14e or the oval or rounded second pocket 16e.

In an alternative embodiment, the thermal pack holding device of the present invention optionally comprises one or more adjustable-length straps (such as are shown in U.S. Provisional Application No. 61/383,417) which are affixed with stitching or removable attachment means such as snaps, buttons, ties, hook and loop closures, or other closure method for extending around the back of the user's body and/or shoulders but without causing added compression of the breasts of the user. A single strap could also be used for holding the thermal pack holding device in place similar to how a neck strap works with eyeglasses to permit the eyeglasses to hang around the neck. The straps may range in number and width such that one, or more than one, strap may be used to hold the device securely in place on the user. In a preferred embodiment, the device of the invention does not comprise straps. The thermal packs 72 and 74 used in the thermal pack holding devices of the present invention preferably are cold packs which fit into each pocket of the device and which rest on each breast. The cold packs may be self-contained bags of crushed ice, ice cubes, gels or liquid, non-toxic refrigerant gel, freezable pellets, or frozen vegetables or any commercially-available self-contained non-toxic cold therapy product. Alternatively, the thermal packs 72 and 74 used herein may be heat packs. Preferably the heat pack material is non-toxic.

The thermal pack holding devices of the present invention are preferably fabricated in two sizes, Basic and Extra Large, although other custom sizes may be constructed. The Basic Size device is approximately 8 inches wide by 21.5 inches long. The Extra Large Size device is approximately 10 inches wide by 25 inches long. However, other sizes ranging from, but not limited to, 5, 6, 7, 8, 9, 10, 11, 12, 13, 14, or 15 inches wide (and fractional widths in between these numbers), to 14, 15, 16, 17, 18, 19, 20, 21, 22, 23, 24, 25, 26, 27, 28, 29, 30, 31, 32, 33, 34, 35, 36, 37, 38, 39, 40, 41, or 42 inches long (and all fractional lengths in between these numbers) are also included herein. In other embodiments, the device of the invention may be wholly or partially constructed of other natural fiber materials or fabrics, synthetic fiber materials or fabrics, such as nylon, rayon, or spandex, or a combination blend of natural/synthetic materials or fabrics, rather than, or in addition to, cotton. In other embodiments, for example wherein alternate materials are used in the construction of the thermal pack holding device of the present invention, materials or fabrics with a rubbery texture or gripping surface may be used for, or on, the base panel of the device so that the gripping side of the base panel faces toward the user's body to reduce shifting of the device while it is in use.

In another embodiment, the device may comprise a pair of concavities for enhancing the fit of the thermal packs about the breasts. For example, shown in FIG. 11, and referred to therein by reference numeral 10f, is an alternate embodiment of the thermal pack holding device of the present invention. Thermal pack holding device 10f is similar to any of devices 10-10e, or other embodiments described herein, in that the thermal pack holding device 10f is constructed to have a base panel 12f having an upper surface 18f, lower surface 20f, a first outer edge 26f, a second outer edge 28f, a first pocket 14f having an inner edge 42f for a first pocket opening 60f, and a second pocket 16f having an inner edge 56f for a second pocket opening 62f. Thermal pack holding device 10f further comprises a pair of concavities (cups) 92 in the base panel 12f, one underneath the first pocket 14f and the other underneath the second pocket 16f. The concavities 92 may be sized and shaped to allow the device 10f to further conform to the shapes of the breasts of the user. The concavities 92 may be formed in the cloth or fabric material of the base panel 12f by any means known to persons having ordinary skill in the act for forming such shapes in cloth or fabric.

An alternative embodiment of the invention is represented by reference numeral 10g in FIGS. 12 and 13. The thermal pack holding device 10g is constructed of the same material and in the same manner as the devices 10-10f described herein except that the openings of the pockets are located generally in a medial (vertically, laterally, or diagonally) portion of the pocket, rather than in or near an edge of the pocket. Any of the embodiments of the invention shown in FIGS. 1-11 or discussed elsewhere in the specification may be constructed such that the opening of the pockets are positioned such as described in FIGS. 12 and/or 13 wherein the device 10g is shown as comprising a base panel 12g, a first pocket 14g, and a second pocket 16g. The base panel 12g has an upper surface 18g, a lower surface 20g, an upper edge 22g, a lower edge 24g, a first outer edge 26g, and a second outer edge 28g. First pocket 14g comprises an upper panel 30g having an edge 42g. The upper panel 30g may comprise a portion of the base panel 12g, or may comprise a separate piece of material attached to the base panel 12g (such as is shown in FIG. 2 for example). First pocket 14g also comprises a lower panel 100g which is attached to the base panel 12g and has a free edge 104g which is parallel to edge 42g of upper panel 30g, and which may be positioned adjacent edge 42g or underneath upper panel 30g. Upper panel 30g and lower panel 100g preferably are each attached to the base panel 12g along all edges except for edges 42g and 104g, respectively, to form an interior space 108g in the first pocket 14g (FIG. 13). Both edge 42g and edge 104g are free substantially along their entire lengths, thus providing a first pocket opening 60g into the interior space 108g of the first pocket 14g, as shown in FIG. 13, for insertion of thermal pack 72 into first pocket 14g.

In a similar manner, second pocket 16g comprises an upper panel 44g having an edge 56g. The upper panel 44g may comprise a portion of the base panel 12g, or may comprise a separate piece of material attached to the base panel 12g (such as is shown in FIG. 2 for example). Second pocket 14g also comprises a lower panel 102g which is attached to the base panel 12g and has a free edge 106g which is parallel to edge 56g of upper panel 44g and which may be positioned adjacent edge 56g or underneath upper panel 44g. Upper panel 44g and lower panel 102g preferably are each attached to the base panel 12g along all edges except for edges 56g and 106g, respectively, to form an interior space 110g in the second pocket 16g (FIG. 13). Both edge 56g and edge 106g are substantially free along their entire lengths thus providing a second pocket opening 62g into the interior space 110g of the second pocket 16g, as shown in FIG. 13, for insertion of thermal pack 74 into the second pocket 16g. First and second pocket openings 60g and 62g may be closeable via any fastening means discussed elsewhere herein for closing any openings discussed herein.

The first and second pocket openings 60g and 62g are shown in FIGS. 12 and 13 as being vertically-oriented. However, in other embodiments, the first and second pocket openings 60g and 62 may be laterally-oriented (i.e., both parallel to upper edge 22g), or may be diagonally-oriented, for example, first pocket opening 60g may be oriented from an upper left hand corner of first pocket 14g to a lower right hand corner and second pocket opening 62g may be oriented from a lower left hand corner of second pocket 16g to an upper right hand corner, or may be oriented in any other diagonal orientation.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, items of manufacture, means, methods and steps described in the specification. As one of ordinary skill in the art will readily appreciate from the disclosure of the present invention, processes, items of manufacture, means, methods, or steps, presently existing or later to be developed that perform substantially the same function or achieve substantially the same result as the corresponding embodiments described herein may be utilized according to the present invention. Accordingly, the appended claims are intended to include within their scope such processes, items of manufacture, means, methods, or steps.

Each of the references, patents, publications or patent documents cited herein is expressly incorporated herein by reference in its entirety.

What is claimed is:

1. A thermal pack holding device, comprising:
a substantially flat base panel having an upper surface, a lower surface, and an outer perimeter, the base panel comprising a soft, flexible fabric material and having a length in a range of about 14 to 42 inches and a width in a range of about 7 to 15 inches, and wherein the ratio of the length to the width is in a range of 2:1 to 4:1;
a first pocket panel attached to or integral to a left portion of the upper surface of the base panel forming a first pocket having a first interior space, and having an opening into the first interior space and wherein the first interior space of the first pocket is sized to contain a first thermal pack; and
a second pocket panel attached to or integral to a right portion of the upper surface of the base panel forming a second pocket having a second interior space, and having an opening into the second interior space and wherein the second interior space of the second pocket is sized to contain a second thermal pack;
and wherein the thermal pack holding device is absent restraining means able to cause, effect, or enhance compression or constriction of the breasts.

2. The thermal pack holding device of claim 1 wherein the ratio of the length to the width is in a range of 2:1 to 3.5:1.

3. The thermal pack holding device of claim 1 wherein the ratio of the length to the width is in a range of 2:1 to 3:1.

4. The thermal pack holding device of claim 1 wherein the first pocket and second pocket each comprise fastening means for closing the openings thereto.

5. The thermal pack holding device of claim 1 wherein the first pocket and second pocket are removably attached to the base panel.

6. The thermal pack holding device of claim 1 wherein the opening into the first pocket is near a right edge portion of the first pocket and the opening into the second pocket is near a left edge portion of the second pocket.

7. The thermal pack holding device of claim 1 wherein the opening into the first pocket and the opening into the second pocket are both along upper edge portions thereof.

8. The thermal pack holding device of claim 1 wherein the opening into the first pocket and the opening into the second pocket are both along medial portions thereof.

9. The thermal pack holding device of claim 1 wherein the base panel has a substantially rectangular shape.

10. A thermal relief system, comprising:
a thermal pack holding device, comprising:
a substantially flat base panel having an upper surface, a lower surface, and an outer perimeter, the base panel comprising a soft, flexible fabric material and having a length in a range of about 14 to 42 inches and a width in a range of about 7 to 15 inches, and wherein the ratio of the length to the width is in a range of 2:1 to 4:1,
a first pocket panel attached to or integral to a left portion of the upper surface of the base panel forming a first pocket having a first interior space, and having an opening into the first interior, and
a second pocket panel attached to or integral to a right portion of the upper surface of the base panel forming a second pocket having a second interior space, and having an opening into the second interior space,
and wherein the thermal pack holding device is absent restraining means able to cause, effect, or enhance compression or constriction of the breasts; and
a first thermal pack disposed within the first interior space of the first pocket, and a second thermal pack disposed within the second interior space of the second pocket.

11. A method of relieving breast pain or discomfort in a subject in need of such relief, comprising:
providing a thermal pack holding device, comprising:
a substantially flat base panel having an upper surface, a lower surface, and an outer perimeter, the base panel comprising a soft, flexible fabric material and having a length in a range of about 14 to 32 inches and a width in a range of about 7 to 15 inches, and wherein the ratio of the length to the width is in a range of 2:1 to 4:1,
a first pocket panel attached to or integral to a left portion of the upper surface of the base panel forming a first pocket having a first interior space, and having an opening into the first interior space and wherein the first interior space of the first pocket is sized to contain a first thermal pack, and
a second pocket panel attached to or integral to a right portion of the upper surface of the base panel forming a second pocket having a second interior space, and having an opening into the second interior space and wherein the second interior space of the second pocket is sized to contain a second thermal pack,
and wherein the thermal pack holding device is absent restraining means able to cause, effect, or enhance compression or constriction of the breasts;
disposing a first thermal pack in the interior space of the first pocket and a second thermal pack in the interior space of the second pocket forming a loaded thermal pack holding device; and
resting the loaded thermal pack holding device upon the breasts of the subject, wherein the subject is in a reclining or semi-reclining position, wherein compression or constriction of the breasts due to restraining means is absent, thereby relieving breast pain or discomfort without causing breast compression or constriction which could lead to cessation of lactation.

12. The method of claim 11 wherein in the thermal pack holding device, the ratio of the length to the width is in a range of 2:1 to 3.5:1.

13. The method of claim 11 wherein in the thermal pack holding device, the ratio of the length to the width is in a range of 2:1 to 3:1.

14. The method of claim 11 wherein the first thermal pack and second thermal pack are cold packs.

15. The method of claim 11 wherein the first thermal pack and second thermal pack are heat packs.

16. The method of claim 11 wherein the first pocket and second pocket of the thermal pack holding device each comprise fastening means for closing the openings thereto.

17. The method of claim 11 wherein the opening into the first pocket of the thermal pack holding device is near a right edge portion of the first pocket and the opening into the second pocket of the thermal pack holding device is near a left edge portion of the second pocket.

18. The method of claim 11 wherein the opening into the first pocket of the thermal pack holding device and the opening into the second pocket of the thermal pack holding device are both along upper edge portions thereof.

19. The method of claim 11 wherein the opening into the first pocket of the thermal pack holding device and the opening into the second pocket of the thermal pack holding device are both along medial portions thereof.
